(19) United States
(12) Patent Application Publication
(10) Pub. No.: US 2004/0203135 A1
(43) Pub. Date: Oct. 14, 2004

(54) Application of b-staged divinylsiloxane-bis-benzocyclobutene for the growth and cultivation of biological materials

(76) Inventors: Jeffrey M Catchmark, Bellefonte, PA (US); Guy P Lavallee, State College, PA (US); James L Murphy IV, Silver Spring, MD (US); Thomas J Manuccia, Silver Spring, MD (US); Lawrence M Dobbs JR., Silver Spring, MD (US)

Correspondence Address: Anthony Colesanti, Colesanti & Associates, Suite1505, 117 North 15th Street, Philadelphia, PA, 19102, US

(21) Appl. No.: 10/486,121

(22) PCT Filed: Aug. 6, 2002
(86) PCT No.: PCT/US02/24895

Related U.S. Application Data
(60) Provisional application No. 60/310,512, filed on Aug. 6, 2001.

(51) Int. Cl. C12M 1/34

(52) U.S. Cl. 435/287.2

(57) ABSTRACT

Benzocyclobutenes have been found to be an ideal material for growing and cultivating biological materials such as biological cells. This property makes benzocyclobutenes suitable for use in devices for analyzing biological materials. A commercially available benzocyclobutene, namely, B-staged divinylsiloxane-bis-benzocyclobutene (BCB) is a spin-on dielectric material produced in several forms by Dow Chemical. This spin-on dielectric may be used in place of other conventional materials requiring use of a more complicated vacuum based deposition process. The applicant has found BCB to be an excellent dielectric material for fabricating microelectrode arrays. In addition, BCB has been found to be an excellent material for growing and cultivating biological

### FIELD OF THE INVENTION

The present invention relates the application of B-staged divinylsiloxane-bis-benzocyclobutene, a spin-on dielectric material produced by Dow Chemical, as a material for growing and cultivating biological materials including biological cells, and as a material fabricating microelectrode arrays for both in vivo and in vitro devices.

### BACKGROUND OF THE INVENTION

Materials useful for the attachment, cultivation and analysis of biological materials, including a wide variety of biological cells, are necessary for conducting research in areas such as medicine, pharmacology, biology, etc. Such materials could also form the basis of a variety of commercial products allowing complex analyses of a wide range of biological materials.

To date, many materials have been explored which could be used, for example, to cultivate or promote the growth of a variety of biological cells. The compatibility of a material to other biological materials is only one part of the requirements necessary for such a material. The material needs to withstand constant exposure to a variety of chemical and biological environments, which may be needed to perform some type of analysis, or just exist due to the nature of the biological system under examination. The material may also need to be resistant to interacting with complex biological systems in applications where a device or structure made from the material would have to be placed in, for example, a human body, for a prolonged period of time. Moreover, the material should be able to be integrated in a variety of manufacturing processes, ideally including those used in the industry for fabricating electronic and biological devices. This would enable the wide spread commercialization of products produced using such a material.

### SUMMARY OF THE INVENTION

B-staged divinylsiloxane-bis-benzocyclobutene (BCB) has been found to be an ideal material for growing and cultivating biological materials such as biological cells. To the applicant's knowledge, BCB is not currently used for this or any related application. Because of this property, though, it is especially useful for the fabrication of devices for the analysis of biological materials. BCB is a spin-on dielectric material typically used in the microelectronics industry for fabricating metal electronic interconnect structures.

According to one aspect of the invention a device is provided for optical, electrical, biological and chemical analysis of biological materials, which includes at least one layer of benzocyclobutene.

Another inventive aspect lies in providing a method for growing and cultivating biological materials, which includes the step of: positioning the biological materials adjacent to a benzocyclobutene.

### BRIEF DESCRIPTION OF THE DRAWINGS

Other aspects, advantages and novel features of the invention will become more apparent from the following detailed description of the invention when considered in conjunction with the accompanying drawings described below:

FIG. 1: A region of a microelectrode array that is about 150 um on a side. There are lines that are the edges of two square 40 um vias (holes) [1 & 2] in the BCB which is covering the Gold electrodes composing the microelectrode array. Two square vias [1 & 2] are completely inside the frame, while the bottom of two more vias [3 & 4] can be seen at the top edge of the frame. At the center of each square via is a small (˜10 um in diameter) circle [5 & 6] which is the actual rnicroelectrode. The irregular, translucent white irregular masses [7] are the C2C12 cardiac myoblast cells. They are obviously thriving. Some are growing on the BCB, some are on the gold, and some span both surfaces. Some appear to be at the point of fusing with other cells to form the usual filamentous structures.

FIG. 2: FIG. 2 shows a single conducting electrode [8] for recording electrical signals from biological samples, fabricated on an insulating substrate [9], covered with an insulating layer of BCB [10].

FIG. 3: FIG. 3 shows a pair of conducting electrodes [8], fabricated on an insulating substrate [9] such as glass, insulated from one another with a layer of BCB [10].

FIG. 4: FIG. 4 shows a multilayer device with several electrodes [8], fabricated on an insulating substrate [9]. Each layer is insulated from the rest by a BCB film [10]. The entire device is then encapsulated with a final layer of BCB [11].

FIG. 5: FIG. 5 shows a single conductor probe for penetrating biological materials, consisting of an electrically conducting core [12] with an insulating coating of BCB [13].

FIG. 6: FIG. 6 shows a probe for penetrating biological materials with a plurality of electrodes [14]. The electrically conductive sites are insulated from one another with layers of BCB [15].

### DETAILED DESCRIPTION OF THE INVENTION

Benzocyclobutenes can be used for growing and cultivating biological materials such as biological cells. This property makes benzocyclobutenes suitable for the fabrication of devices used for analyzing biological materials. Examples of benzocyclobutene derivatives may be found in U.S. Pat. Nos. 5,334,773; 4,788,187; and 3,408,391, all of which are specifically incorporated herein by reference.

One example of a commercially available benzocyclobutene useful for both growing and cultivating biological materials as well as for fabricating devices for analyzing biological materials is B-staged divinylsiloxane-bis-benzocyclobutene (BCB). BCB is a spin-on dielectric produced in several forms by Dow Chemical. This spin-on dielectric may be used in place of other conventional materials requiring use of a more complicated vacuum based deposition process.

The applicant has found BCB to be an excellent dielectric material for fabricating microelectrode arrays. Unexpectedly, BCB also has been found to be an excellent material for growing and cultivating biological cells. Particular compositions of BCB examined included Dow Chemical Composition Nos. 4022-35, but the other compositions are expected to exhibit identical or very similar results due to the nearly identical chemical and physical properties of these BCB materials once cured. Curing is done to convert the liquid BCB to a useful dielectric film.

In addition, the BCB used to fabricate the microelectrode arrays was found to be resistant to degradation, including delamination and separation of single and multi-layer films, when exposed to corrosive environments. The low moisture absorptivity of the BCB means that it is especially suitable for application where it will be exposed to water based solutions involving biological materials.

Fabrication of BCB Samples In order to perform basic feasibility testing of the biocompatibility of BCB based devices, we designed a series of test samples. In this case, 2 metal conductors composed of a 5 nanometer evaporated layer of Titanium followed by a 50 nanometer evaporated layer of Gold were patterned on a BCB coated glass substrate. The contacts were 40 microns wide and several centimeters long. The spacing between the contacts was 20 to 100 microns. The contacts (and glass substrate) were coated with BCB and cured using a process similar to that proposed by Dow Chemical, the supplier of BCB. Several devices were fabricated, including structures to test for delamination, absorption of saline, and biocompatibility. Completed wafers were diced, and the dies cleaned and prepared for testing.

### Biocompatibility Tests of BCB Samples

One series of tests looked for any negative effects on the growth and adhesion of cultured myoblasts by BCB itself. BCB samples as well as control plates were seeded with C2C12 mouse myoblast cells from a line established from normal adult C3H mouse leg muscle. Muscle cells were chosen for their ease of cultivation. The samples were seeded at a density of 2×10\hat{ }4 cells/cm\hat{ }2 and cultured in in Dulbecco's Modified Eagles Medium with 4 mM Glutamine, 4.6 g/L glucose, 1.5 g/L Na2HCO3, 1.0 mM pyruvate and supplemented with 10% heat inactivated horse serum for 30 hours at 37 C. in a humidifed 5% CO2/air incubator. The cells were fixed with 10% Formalin in PBS for 15 minutes and washed with PBS several times before preparation for photography.

The samples were photographed using a Phillips CCD camera coupled with an American Optical Phase-Star fluorescence microscope. A PC equipped with an ATI video capture card was used to store the images. A 10× objective was used for all photographs.

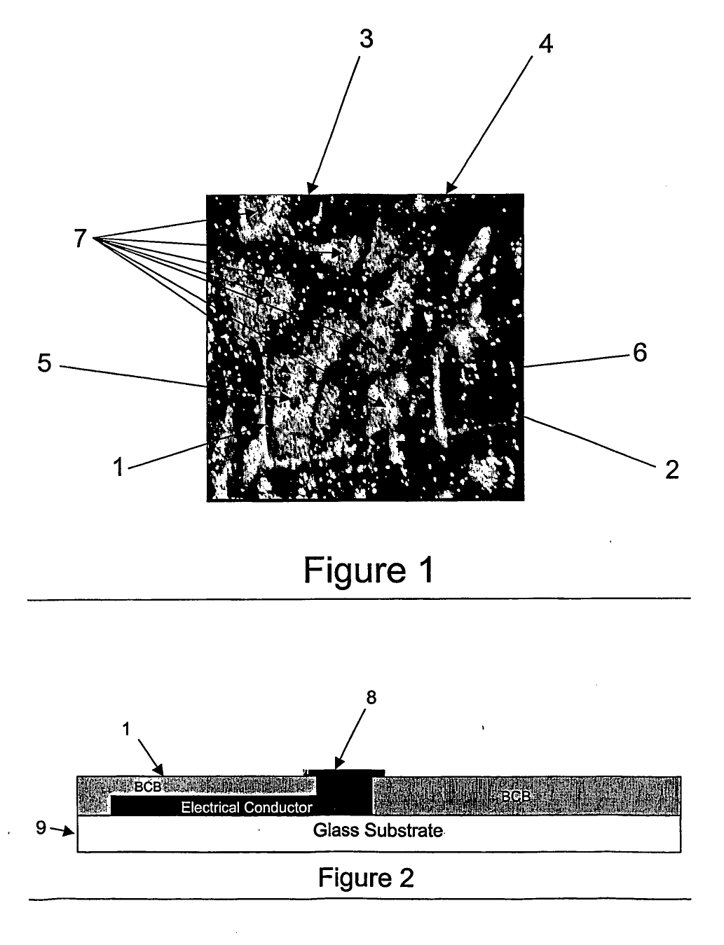

FIG. 1 shows four electrodes with several cells in close proximity, and process formation between some pairs. The large rectangular outlines are the edges of the vias opened in the BCB layer, while the smaller outlines delineate the edges of the Au electrodes. Thin black lines have been hand-drawn to help identify the features. The smaller electrodes are 12.5 μm square and the large ones 25 μm. The BCB openings are 100 μm wide. The sample was trans-illuminated using phase contrast optics which highlights the cells, as well as the edges of the vias and electrodes. Cells are growing normally over the entire sample, including the gold electrodes, but cannot be seen over the Au because of its opacity and the method of illumination. In these initial experiments, the cells appear to exhibit no preference for either the BCB or the gold surface.

In these initial screening experiments, no difference was noted between BCB and control substrates with respect to cell growth, adhesion, morphology or process formation/fusion.

### Adhesion Testing of BCB Samples

In order to examine the long term mechanical durability of the BCB film we immersed samples in a saturated saline solution maintained at 75° C. No delamination or other changes were seen after several days' immersion. We then increased the temperature of the saturated salt solution to just below its boiling point, and maintained it there for another week. Again, there was no evidence of problems. In our experience, many other polymer films, and even BCB films prepared in less than optimum conditions would have delaminated in less than a day at 75° C. This preliminary accelerated aging test provides evidence that BCB is very resistant to attack by saline and should perform well even after long periods in solution at more reasonable temperatures and concentrations.

### Evaluation of Electrical Performance in Saline

Another potential problem with electrical devices immersed in biological media is shorting of the insulating layers (i.e., BCB) due to volumetric absorption of salt water or microscopic delamination followed by influx of bulk water. BCB is specifically designed to resist absorbing water, exhibiting less than 0.25% uptake at 85% relative humidity. As our samples are designed to be immersed in saline solutions, we elected to perform our own testing to confirm the resistance of BCB to absorb water.

The test configuration consisted of pairs of conductive traces buried under a 2 μm thick BCB layer. The trace separation varied from 4 μm to 100 μm. During testing, the chose pair of traces was connected to a Keithley Model 600A Electrometer in order to measure the inter-trace resistance. Prior to testing the resistance for all samples was greater than 4×1012.

The first part of the test involved immersing the test structure in a dilute saline solution (0.171 mol/L) maintained at a temperature of 35° C. No decrease in resistance from an initial value of approximately 4×10\hat{ }2 ohms was seen after several days exposure. As in the delamination test, we then increased the concentration and the temperature to the boiling point. Under these conditions we did see a small, repeatable decrease in resistance down to about 2×10\hat{ }12 ohms, which was reversible upon cooling the solution. Our interpretation is that we were measuring an extremely small reversible solubility of water in the 1 μm BCB under truly extreme conditions. To appreciate what stunningly high resistances we are dealing with, condensation from breath on the unencapsulated region of the test structure immediately decreases the resistance to under 1×10\hat{ }6 ohms.

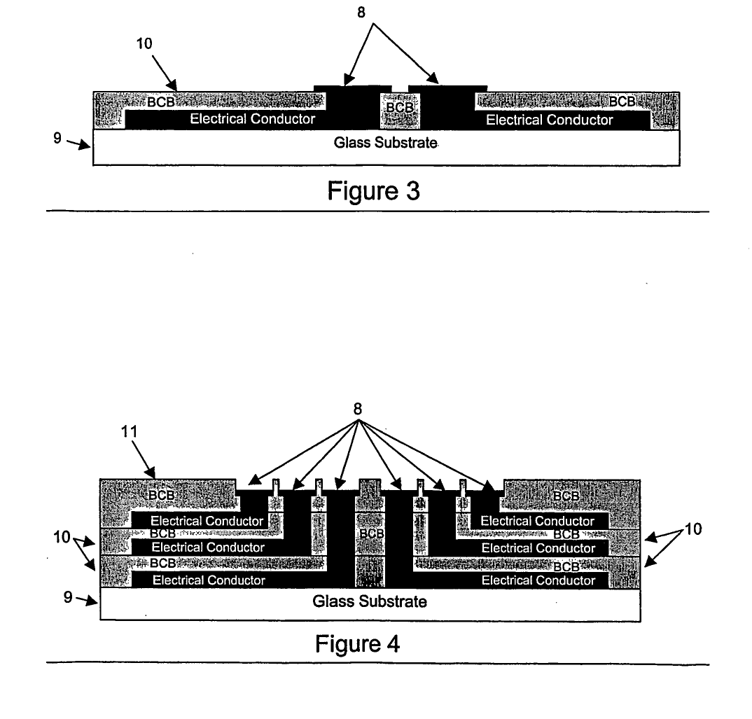

This work clearly demonstrates the ability to use BCB as a material for the cultivation and growth of biological cells and for the fabrication of devices to analyze the optical, electrical, biological and chemical properties of biological materials. Many other related biological and chemical applications of this material will become apparent to anyone skilled in the art. Devices made from BCB may consist of one or more layers of the BCB polymer and a conductive layer. See FIGS. 2 through 6.

Devices made from BCB have in vitro and in vivo uses. A specific in vitro or in vivo use is as a microelectrode array for the analysis of the electrical, biological and chemical properties of biological materials, including biological cells. Microelectrode arrays consist of arrays of conductive electrodes separated by a passivation layer and are often used for recording from and stimulating biological cells, specifically neuronal tissue. See FIGS. 2-4.

On devices made from BCB the surface of the BCB that is exposed to a biological material may be chemically or physically modified such that it has an enhanced ability to absorb a protein layer on said applied layer when exposed to a biological fluid, and wherein cell attachment, mass cell culture, cell growth or mass tissue culture on said applied layer is enhanced. This protein layer may be but is not limited to one or more members of the following traditional biological cell growth and adhesion promoters: fibronectin, laminin, polylysine, neurotrophins and vitronectin.

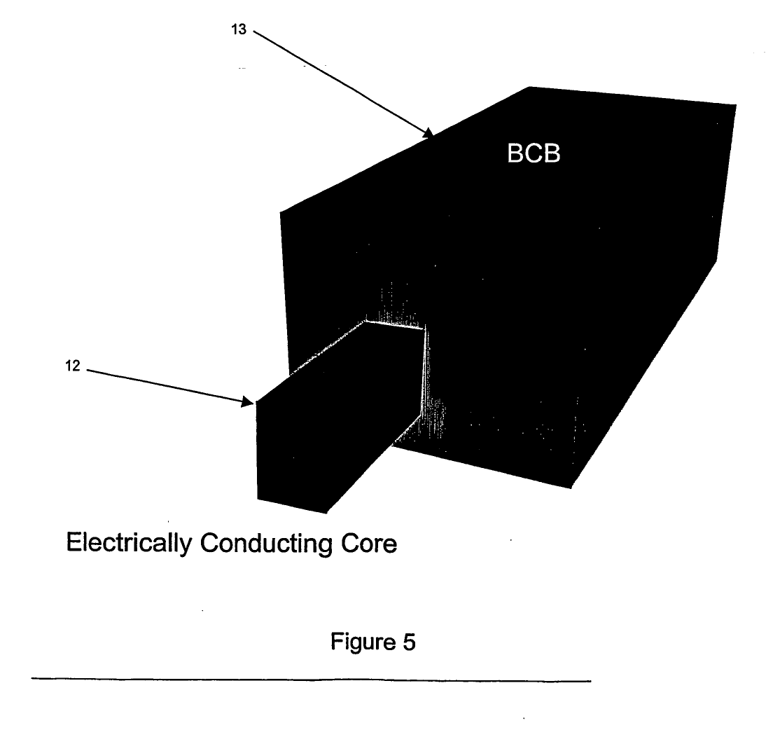

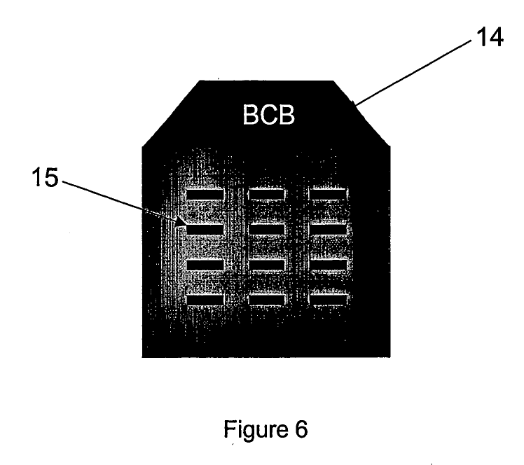

Devices made of BCB may be made into a probe designed to penetrate biological materials. See FIGS. 5 and 6. This type of device and the abovementioned microelectrode array may be used as a neural prosthesis. This prosthesis may be but is not limited to one or more of the following types of neural prostheses: cochlear, retinal, visual cortex, pain control, bladder and other sphincter control; posture, balance and other gait prosthesis.

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is the following:

1. A device for optical, electrical, biological and chemical analysis of biological materials comprising:
at least one layer of a benzocyclobutene.

2. The device of claim 1 wherein the benzocyclobutene is a B-staged divinylsiloxane-bis-benzocyclobutene (BCB).

3. The device of claim 2 wherein said device comprises a plurality of BCB layers.

4. The device of claim 2 wherein said device is a microelectrode array.

5. The device of claim 2 wherein said device is a probe for penetrating biological materials.

6. The device of claim 2 wherein said device is a neural prosthesis.

7. The device of claim 4 wherein said prosthesis is one of a cochlear, retinal, visual cortex, pain control, bladder and other sphincter control, posture, balance and other gait prosthesis.

8. In a system for optical, electrical, biological and chemical analysis of biological materials the improvement which comprises using BCB as a contact surface for the biological materials.

9. An apparatus for growth and cultivation of biological materials comprising: at least one layer of benzocyclobutene.

10. The apparatus of claim 9 wherein the benzocyclobutene is a B-staged divinylsiloxane-bis-benzocyclobutene (BCB).

11. The apparatus of claim 9 comprising a plurality of BCB layers.

12. The apparatus of claim 9 wherein at least one of the at least one layer of BCB is modified such that it has an enhanced ability to absorb a protein layer when exposed to a biological fluid, and wherein cell attachment, mass cell culture, cell growth or mass tissue culture on said at least one modified layer is enhanced.

13. The apparatus of claim 12 wherein the absorbed protein layer comprises at least of fibronectin, laminin, polylysine, neurotrophins, and vitronectin.

14. In an apparatus for the growth and cultivation of biological materials the improvement which comprises using BCB as a contact surface for the biological materials.

15. A method for growing and cultivating biological materials comprising the step of:
positioning the biological materials adjacent to a benzocyclobutene

16. The method of claim 13 wherein the benzocyclobutene is a B-staged divinylsiloxane-bis-benzocyclobutene (BCB).
